(19) United States
(12) Patent Application Publication
(10) Pub. No.: US 2007/0141119 A1
(43) Pub. Date: Jun. 21, 2007

(54) Use of additionally fermented distillers grains for preventing and/or treating increased blood sugar values

(75) Inventors: Karl-Heinz Zirzow, Rudelzhausen (DE); Dirk Bokenkamp, Werne (DE); Martin Muller, Freising (DE); Tom Dieck Heike, Freising (DE)

Correspondence Address: FITCH EVEN TABIN AND FLANNERY, 120 SOUTH LA SALLE STREET, SUITE 1600, CHICAGO, IL, 60603-3406, US

(73) Assignee: Bioghurt Biogarde GmbH & Co., KG, Freising (DE)

(21) Appl. No.: 10/579,019

(22) PCT Filed: Nov. 12, 2004
(86) PCT No.: PCT/EP04/12875

§ 371 Date: Jan. 16, 2007

(30) Foreign Application Priority Data
Nov. 12, 2003 (DE) 103 52 822.9

(51) Int. Cl. A61K 35/74; A61K 47/00

(52) U.S. Cl. 424/439; 424/93.45

(57) ABSTRACT

The present invention relates to the use of distillers grains fermented with a yogurt culture and/or buttermaking culture for producing a composition for preventing and/or treating increased blood sugar values.

### DESCRIPTION

The present invention relates to the use of distillers grains fermented with a yogurt culture and/or butter culture for producing a composition for preventing and/or treating increased blood sugar values.

Distillers grains contain numerous nutritionally important components such as residual carbohydrates, fats, dietary fibers etc., so that they have long played an important role as feedstuff in animal husbandry, and in particular in pig raising.

Especially the dietary fibers and vitamins still present in the distillers grains fermented using yeast cultures (“mashed”) make this original “waste material” a valuable secondary raw material, not least also for human nutrition.

For instance, WO 01/10 245 discloses a method for producing foods, dietetic foods and food additives, which method starts from distillers grains taken directly from the still, that is distillers grains fermented using yeast. After the distillers grains have been thickened, but not dried, they are fermented a further time with milk and a yogurt culture and/or buttermaking culture. In this method, essentially four method steps are carried out in succession, first
a) the distillers grains being thickened to approximately 15 to 17% DM (dry mass) in a special pressure range, then
b) the distillers grains being mixed with a milk inoculated with the yogurt cultures or butter-making cultures, and subsequently
c) being fermented either with the yogurt cultures at 38 to 48° C. for 10 to 20 hours, or with a butter-making culture at 18 to 24° C. for up to 36 hours; subsequently
d) this preproduct, mixed with further-milk or a milk concentrate, is cooled to below 15° C.; optionally an additional spray-drying step follows, in order to arrive at a pulverulent and storable product.

The products obtainable by this method, not only as stable liquid product, but also as storable powder, are nutritionally valuable whole grain and protein products from which the starch fractions have been virtually completely removed, with a dietary fiber fraction remaining high on account of the gentle and additional fermentation, a residual starch content of at most 4% by weight being achieved.

In connection with the nutritional properties of whole grain and, in particular, of whole grain rice, in a comparison with polished products, it was found that they are able to improve significantly the insulin sensitivity and blood fat values (Jang Y. et al.: “Consumption of Whole Grain and Legume Powder Reduces Insulin Demand, . . . , in Patients with Coronary, Artery Disease: Randomized Controlled Clinical Trial”, Arteroscler. Thromb. Vasc. Biol. 21:2065-2071, 2001).

JP 101 46 166 discloses physiologically active compositions which can comprise, inter alia, brewers grains or aqueous extracts thereof. These grains fermented exclusively with yeasts are used for treating cancer and associated symptoms, inter alia, diabetes and obesity also being mentioned.

According to JP 2002-371003, concentrated extracts of a fermented wheat material are said to be suitable for treating diabetes, with them being said in particular to prevent the increase in blood sugar level. This antidiabetic effect was demonstrated in mice populations using a wheat extract exclusively fermented using yeast.

Especially type II diabetes and associated (pre) forms thereof are playing an important role in terms of health policy, but also society, in the industrialized nations, but also, owing to social and cultural changes, increasingly in developing countries and in the younger population, already at present, but especially in the future.

Diabetes, for a long time, has no longer been only a harmless accompaniment of aging, but it is a serious metabolic disorder which leads to life-shortening secondary disorders, such as, for example, heart attack, stroke, kidney failure, blindness or what are termed the “sugar-related” amputations.

From the social point of view, these complications caused by diabetes and feared, especially, give rise to high costs. It is of concern, in particular, not only to recognize promptly the insulin resistance manifesting in type II diabetes and preceding this syndrome by many years, which increases with advancing age, but also to treat it and thus prevent the typical diabetes symptoms.

For the present invention, the object, therefore, was to provide a composition which, on account of its, in particular, nutritional properties, is suitable for preventing pathologically increased blood sugar values and/or to lower already-increased blood sugar values. The use of a substantially natural composition was in the foreground, which composition is readily accessible and where possible, in addition to the effects actually sought, is also able to exercise other beneficial effects on the metabolism of the body.

This object has been achieved by the use of distillers grains which, in addition to a fermentation with yeast, were also fermented with a yogurt culture and/or buttermaking culture, for producing a composition for preventing and/or treating increased blood sugar values.

Distillers grains are the liquid residue of the fermentation of carbohydrate-containing raw materials from cereals, after removal of the alcohol by distillation. They contain the unfermentable constituents of the mashed raw material and residues of the yeast cells. 100 kg of fermented cereals produce approximately 500 l of distillers grains containing 5 to 7% dry residue (pH 3.6-4.0). About half of the dry residue consists of soluble constituents, about half of insoluble constituents.

Surprisingly, it has been found, in the regular inventive use, that increased blood sugar values can be brought significantly to lowered values remaining constant.

For the valuation of elevated blood sugar values, a distinction is made between fasting and/or postprandial (2-hour-)blood sugar value. In the case of fasting blood sugar values ≧110 mg/dl, an increased blood sugar value is spoken of, and in the case of fasting blood sugar values ≧110 mg/dl and <126 mg/dl, a pre-diabetes form is present, and in the case of fasting blood sugar values ≧126 mg/dl, diabetes is present. In the case of postprandial blood sugar values ≧140 mg/dl, an elevated blood sugar value is spoken of, and in the case of postprandial blood sugar values ≧140 mg/dl and <200 mg/dl, a prediabetes form is present, and in the case of postprandial blood sugar values ≧200 mg/dl, diabetes is present.

In connection with the present invention, the use of distillers grains fermented completely using yogurt cultures and/or butter cultures has proved particularly suitable, in particular preferably distillers grains having a residual starch content of a maximum of 4% by weight, based on the total weight of the distillers grains.

A preferred variant found is the use of the inventive distillers grains for producing a composition for preventing, and/or treating patients having, prediabetes and/or diabetes and/or a predisposition thereto, and associated forms and/or preforms. These are customarily preferably diabetes type IA and IB, type IIA and IIB and/or autosomally inherited diabetes of youth.

Prediabetes is a stage having usual glucose tolerance without clinical symptoms such as hyperglycemia, glycosuria etc., which can precede the outbreak of Diabetes mellitus by years, The indication of prediabetes is not necessarily based on retrospective diagnosis, but it is at any rate usually based on the time period between conception and recognition of diabetes.

Type I diabetes, or insulin-dependent diabetes, is a genetically predisposed form of diabetes with gradual exhaustion of the body's insulin secretion up to absolute lack of insulin. In the case of type IA, which occurs in practice in childhood, viral infections are presumed to promote manifestations. In the case of type IB of adults, which occurs up to the age of 35, islet cell antibodies are frequently found in the serum. This type frequently also occurs together with other autoimmune diseases. Therapy consists of a diet and also insulin substitution.

Type II diabetes, or insulin-independent diabetes, usually occurs at an older age, frequently occurs in families, and is apparently genetically caused. The rare type IIA includes normal-weight patients, whereas most patients belong to type IIB, and are overweight. In diabetes type II, there is restricted, maintained or occasionally even increased insulin secretion, and also reduced insulin sensitivity of the tissues and thus relative lack of insulin, as far as absolute lack of insulin in later stages. Therefore, in the late stage of type II diabetes, insulin therapy is definitely frequently met. Therapy used for treatment is weight reduction, diet and possibly oral antidiabetic drugs. The autosomally inherited diabetes of youth, also MODY (maturity onset diabetes of the young) has a different, generally clinically mild, course without late complications. It is an independent diabetes form which typically occurs before the age of 25 and is accompanied by increased insulin secretion, peripheral insulin resistance, and obesity. Therapy used for this diabetes type is weight reduction. Therapy using insulin and oral antidiabetic drugs is not necessary, but lifelong metabolic controls are necessary.

The inventive distillers grains are suitable for therapy of said diabetes types, since in the latter lowering of the blood sugar values is necessary and as low a stress as possible of the blood sugar balance is sought.

In addition, the distillers grains are preferably used according to the invention for preventing and/or treating diabetes-associated forms and/or preforms. These include the secondary diabetes forms, potential diabetes, latent diabetes, decreased glucose tolerance and/or clinically manifest diabetes.

Potential diabetes is present in persons having a non-pathological oral glucose tolerance test, for whom diabetes can be predicted with high probability owing to a familial burden.

Latent diabetes is present in persons

1. having normal values in the oral glucose tolerance test who display pathological values under burdens such as pregnancy, infection, stress or after increase in weight (adiposity),

2. who exhibit pathological blood sugar curves in provocation tests.

It is possible to speak of a decreased glucose tolerance in persons

1. having pathological values in the oral glucose tolerance test, and for whom the fasting blood sugar is below 110 mg/dl,

2. who exhibit pathological values in the oral glucose tolerance test and for whom the fasting blood sugar values are ≧110 mg/dl and <126 mg/dl,

3. whose postprandial blood sugar values are ≧140 mg/dl and 200 mg/dl.

Clinically manifest diabetes is present in patients having pathological blood sugar values and urea sugar excretion, and also in the case of predominantly typical symptoms and possible complications of diabetes.

The secondary diabetes forms include pancreas disorders, e.g. hemochromatosis, endocrinopathies, e.g. acromegaly, drug-caused diabetes, e.g. steroid diabetes and other forms of decreased glucose tolerance and/or pregnancy diabetes. In pregnancy diabetes, the pregnancy acts in the sense of a promotion of the manifestation, or, in some circumstances, impairs the metabolic position in already manifest Diabetes mellitus. It is an insulin-requiring diabetes form. After birth, the insulin requirement decreases markedly. Since in these diabetes-associated forms and/or preforms, likewise a lowering of the elevated blood sugar values is therapeutically indicated, the inventive use of the fermented distillers grains is suitable for preventing and/or treating these forms and/or preforms.

In the context of the present invention, the use in the case of patients having type II diabetes (diabetes type 2) and associated forms and/or preforms has been found particularly suitable.

Overall, it is likewise possible to use the special fermented distillers grains according to the invention for reducing body weight, particularly preferably for simultaneous reduction of body weight which is expressed particularly in a significantly improved body mass index (BMI) of >1 unit per 4 weeks.

In this connection, the present invention takes into account a preferred use variant for producing a composition for preventing, and/or treating patients having, adiposity, and particularly preferably for preventing, and/or treating patients having, adiposity, and simultaneously increased fasting blood sugar values. By means of the inventively used distillers grains, it is possible to treat successfully these obese persons having simultaneously increased fasting blood sugar values gently and without drastic intervention in their nutritional habits, since the effect of the product goes beyond the effect caused by simple weight decrease.

The combination of these advantageous properties of the multiply fermented distillers grains used was thus not to be predicted.

The fermented distillers grains in the context of the present invention thus preferably relate not only to the therapeutic use, but also to the non-therapeutic use.

As a further preferred variant, the inventively used distillers grains can be used for preventing, and/or treating patients having, metabolic syndrome. A person, according to a WHO definition (World Health Organization (1999), Definition, diagnosis and classification of diabetes mellitus and its complications. Part 1, Diagnosis and classification of diabetes mellitus. WHO: (WHO/NCD/NCS/99.2)), has a metabolic syndrome when, in addition to detection of insulin resistance, they meet two of the following criteria:
hypertension defined as antihypertensive therapy and/or blood pressure >160/90 mmHg,
dyslipidemia defined as increased plasma triglycerides (≧1.7 mmol/1) and/or reduced HDL cholesterol (<0.9 mmol for men and <1.0 mmol for women),
adiposity BMI (30 kg/m²) and/or a waist/hip circumference quotient >0.85 for women and >0.9 for men,
microalbuminuria (albumin excretion rate 20 μg/M).

If a person has type II diabetes or decreased glucose tolerance, then he or she has a metabolic syndrome if two of the abovementioned criteria are met.

Particular preference is given to the inventive use for preventing and/or treating patients having, insulin resistance, arteriosclerosis, lipid, protein and/or carbohydrate metabolic disorders. Surprisingly, in the case of regular use according to the invention, it has been found that, especially in the case of persons having a predisposition, the insulin sensitivity can be markedly and permanently improved, not only the blood sugar level, but also the insulin level being kept to an extremely low value. In this case the distillers grains have been found to be virtually insulin neutral, so that the insulin hunger spiral was set in motion only to an extremely low extent.

In addition, there was the observation, which was likewise not foreseeable, that the improvement of the metabolic syndrome factors, to be considered as critical in connection with diabetes, can be achieved particularly even without an otherwise customary strict reduction diet, which is thought to be due to the extremely low glycemic index (GI) of the distillers grains used. The glycemic index classifies carbohydrate-containing foods according to their blood-sugar-elevating action. It expresses in numbers the blood-sugar-elevating action of the carbohydrates or carbohydrate-containing foods. The blood-sugar-elevating action of glucose serves as a reference and has been assigned the reference value 100. The GI is defined as the area under the curve of the blood sugar values, that is a GI of 50 means that the rise in blood sugar of this product is only half of the rise in blood glucose above the fasting value. A high glycemic index is defined as between 70 and 100, a medium glycemic index between 55 and 70, and a low glycemic index below 55. In addition, a lowering of fructosamine, blood pressure and also the blood fats triglycerides and cholesterol has been achieved.

The use according to the present invention is based in particular on using specially produced distillers grains, as described in WO 01/10245. It is therefore proposed for preference that according to the invention distillers grains are used which were produced by the distillers grains being taken off directly from the still, then thickened, but not dried, and subsequently being fermented with milk and a yogurt culture and/or buttermaking culture.

It has been found to be particularly suitable to make use in this case of distillers grains which were produced by
a) raw distillers grains prefermented with yeast cultures, usually in the context of alcoholic fermentation, being thickened to approximately 15 to 17% DM (dry mass) at a pressure of 100 to 800 mbar, then
b) being mixed with a milk already inoculated with the yogurt culture and/or buttermaking culture,
c) this mixture either being fermented with a yogurt culture for 10 to 20 hours at a temperature of 38 to 48° C. or this mixture being fermented with a buttermaking culture for up to 36 hours at a temperature of 18 to 24° C., and thereafter
d) cooled, mixed with further milk or milk concentrate, to temperatures below 15° C., and
e) which is optionally subsequently spray dried.

In this manner a high-fiber, protein-containing whole grain product is obtained, the cultures of which, e.g. in a non-heated, but sterile liquid formulation, exhibit probiotic properties. In addition, the distillers grains which are multiply fermented in this manner contain essential fatty acids, vital vitamins, minerals and trace elements. As a result of the downstream spray drying, the cultures used lose their viability, which makes the product extremely stable and storable. Even a moisture content possibly increasing during storage cannot restart the fermentation in this manner.

The resultant products can obviously be provided with further, and in particular, fermentable, additives, which additionally beneficially affect the properties of the end product. For instance, it has been found that, for example, honey, lecithins and/or soya protein can enter into complex compounds with distillers grains. Chemical/physical interactions between the lecithins, the protein and the individual groups of substances of the distillers grains form in this manner nutrient systems which differ considerably from those of the individual components with respect to their chemical, physical and biological behavior. Obviously, simple mixing not only of the spray-dried product, but also of the non-spray-dried product, with minerals, vitamins, flavorings, aroma substances and colorings is also possible, which additionally increases the product variety.

It must be emphasized that all the possible products based on distillers grains possess a specific quality with respect to flavor and odor which is no longer reminiscent of the typical raw distillers grains, and which is therefore very readily accepted by consumers, which is expressed, in particular, in increased compliance.

In principle, the use in the claimed context is not restricted to any special starting material, but distillers grains based on wheat, barley, rye, oats, corn and/or rice are recommended.

To achieve the sought-after effects, the distillers grains used should be administered in the context of the present invention in a daily dose of 100 to 400 g, preferably 150 to 300 g, and particularly preferably 200 to 250 g, 2 to 5 individual doses per day, and in particular equally sized individual doses, being particularly recommended.

As ideal administration form which has been found for the distillers grains are those in viscous form, with these particularly preferably being administered mixed into water or fermented milk, and very particularly preferably into curdled milk, buttermilk, yogurt, quark or kefir.

If desired, the taste of the preparation can also be modified by sweeteners, for example fructose (sugar replacer), cyclamate or saccharine. However, they should not contain domestic sugar (sucrose), barley sugar (glucose) and malt sugar (maltose). Although the distillers grains can also be mixed into fruit teas or fruit juices or drinks containing these, fruit juices, in contrast to fruit teas, usually contain sugars which, via an insulin rise, would impair fat combustion. Overall, it is necessary to ensure that the mixing media used contain neither too much sugar, nor too much fat.

The present invention likewise provides administering the distillers grains as a meal replacement and/or meal supplement, which particularly preferably should proceed over a period of at least 3 successive weeks If the feeling of wellbeing permits, the inventive use can also particularly preferably be performed without restriction as to time limit.

If desired, or medically advised, the inventive use can also proceed in combination and/or together with further anti-diabetes acting or diabetes-preventing active ingredients, preferably active ingredients counteracting or preventing type II diabetes, which additionally reinforces the desired result.

Particularly preferably, the proposed use is in the form of, and/or in combination with, and/or together with, food supplements, functional food and/or special diet.

Food supplements are foods which, because of their nutritional value, are consumed to supplement the daily customary diet of healthy persons whose supply of one or more nutrients from this customary diet is marginal, doubtful or inadequate. The purpose is sufficient supply of the body with nutrients or other nutritionally active substances such as vitamins, minerals including trace elements, essential fatty acids, amino acids, proteins and/or carbohydrates. “Functional foods” are defined as foods which have a specific additional benefit which goes beyond the nutritional benefits of the nutrients present therein. This additional benefit is achieved by adding certain substances or by changing the original properties, for example by genetic engineering. The substances most frequently added in this case are vitamins, trace elements, probiotics and prebiotics, certain fatty acids or fat substitutes, secondary plant constituents or enzymes.

Special diet encompasses, for example, tube-feeding diet, sterile food, milk-free special diet for infants, low-lactose allergen special diet for feeding infants etc. As a result of this proposed use in the form of, and/or in combination with, food supplements, functional foods and/or special diet, the patient compliance can additionally experience a marked increase and the advantageous activity of the distillers grains can be reinforced and/or supplemented by other active ingredients.

This is the case, in particular, when use is made of the distillers grains in the form of, and/or in combination with, and/or together with, creatine, creatine monohydrate, α-lipoic acid, phytosterol, poly-unsaturated fatty acids and/or phospholipids and also their derivatives and/or mixture. Particular preference is given to the use of polyunsaturated fatty acids, for example docosahexaenoic acid (DHA), eicosapentaenoic acid (EPA) and/or conjugated linoleic acid (CLA), and also phospholipids, e.g. lecithin or phosphatidylserine.

In the context of the inventive use of special multiply fermented distillers grains, the body can be supplied with all essential macro- and micronutrients. The proteins present in the distillers grains supply and protect the muscle tissue and, on account of the specific composition of distillers grains and their use as meal replacement, predominantly fats are consumed, which, in addition, are optimally combusted, in interaction with the components present in the distillers grains.

An additional aspect is that the use of the multiply fermented distillers grains exerts a regulating effect on the intestinal flora. Particularly preferably, the use of the specific, soluble and insoluble dietary fibers of the distillers grains exhibits a detectably beneficial and regulating effect on the intestinal flora, which comes into effect beneficially in particular in the use of distillers grains as a meal replacement and/or meal supplement in the case of sufficient consumption of liquid.

The examples hereinafter explain the advantages of special multiply fermented distillers grains in the inventive use.

### EXAMPLES

A controlled, open, randomized cross-over study was carried out in which the insulin sensitivity was studied in subjects having adiposity and simultaneously increased fasting blood sugar (prediabetics). Use was made of double-fermented distillers grains, produced according to WO 01/10 245, having a high fraction of starch-reduced whole grain wheat, which is marketed under the trade name “Vibamin®”.

The study period was, for each participant, approximately 10 weeks, with the subjects in the cross-over method each being treated for 4 weeks with “Vibamin®” or with a commercially obtainable comparison product in accordance with a worked out treatment plan; between the individual treatment periods, 2 weeks were provided for the “wash-out” phase. As a comparison for the use of “Vibamin®”, use was made of a product counted among the market leaders, which can be obtained on the open market as a meal drink for weight reduction.

In total, 30 participants aged between 18 and 70 years, whose body-mass index (BMI) was >29 and <40 kg/m², participated in this open, randomized, duplicate “cross-over study”. The fasting blood sugar values at the start of the study were between 110 and 126 mg/dl. An additional anti-diabetic treatment with tablets or insulin did not take place. In addition, no serious existing diseases were observed in the subjects.

Two daily (main) meals were provided by feeding at least 45 g of each of either “Vibamin®” or the comparison product, and the remaining main meal was consumed as otherwise conventional. It was left open to the subjects to consume, at any time of day, snacks consisting of “Vibamin®” or the comparison product.

It was necessary, in any case, to take into account the fact that in the subjects of the study, under “Vibamin®” or the comparison product, there was a slight reduction in the daily calorie supply (guide value of a balanced diet approximately 1700 kcal/day).

At the start of the study, an extensive dietetic consultation was carried out in which the dietetic concept was explained to the participants.

After one week of the diet, both with “Vibamin®” and with the comparison product, there was an interim visit in which the participants were questioned about their personal experience with the diet and the actual body weight was determined. In supplementation, the same parameters were gathered as at the visits at the start and end of the treatment.

The subjects kept a standardized diary during the study, in which the time of day and amount of “Vibamin®” or comparison product consumed were recorded.

The compliance of the participants was increased by unannounced telephone calls for the subjects of the study.

The foreground of the study was occupied by the change in insulin resistance index HOMA-IR (ROMA-Index) after 4 weeks diet in each case with “Vibamin®” or the comparison product in prediabetic subjects (subjects having adiposity and simultaneously increased fasting blood sugar). In this case, the absolute improvement of the HOMA-Index was of interest, but of still more interest was the relative improvement of the HOMA-Index after cancellation of the effect caused by weight loss. In this way it is possible to determine the effect solely caused specifically by the treatment.

Likewise, the following were recorded: the change in body weight after 4 weeks on each diet, the change in fructosamine content, fasting blood sugar, fasting insulin, waist-to-hip ratio, triglycerides and cholesterol, and also the blood pressure. The subjective feeling of wellbeing and the postprandial satiation feeling after 4 weeks in each case were likewise recorded using a standardized questionnaire.

In the subsequent result of this study, the significant lowering of the fasting blood sugar level in all subjects is to be emphasized in particular. Likewise, without exception, in all treated subjects, a drastic and continuing weight reduction (detected, inter alia, by BMI) was observed. However, it should be emphasized for the “Vibamin®”-treated subjects that the improvement of the fasting blood sugar, the fasting insulin and the main parameter HOMA-Index, even after elimination of the interfering parameter “weight”, remained significant (p=0.005; p=0.03 and p=0.007), whereas in the comparison product, only the weight reduction caused the improvement.

No adverse effects on other vital signs, such as blood pressure, pulse or ECG, were observed either under “Vibamin®” or under the comparison product,

Three of the subjects terminated the study prematurely at their own request.

TABLE 1
HOMA-IR (with p-values absolute)

| Com- | | | | | | | |
|---|---|---|---|---|---|---|---|
| parison | | | | | | | |
| product | Be- | Af- | Differ- | Vibamin | | Af- | Differ- |
| Subject | fore | ter | ence | Subject | Before | ter | ence |
| 301 | 3.3 | 3.39 | −0.09 | 301 | 4.82 | 3.53 | 1.3 |
| 302 | 3.85 | 3.06 | 0.79 | 302 | 3.92 | 1.14 | 2.78 |
| 303 | 3.22 | 2.44 | 0.77 | 303 | 5.77 | 2.84 | 2.94 |
| 304 | 3.44 | 1.79 | 1.65 | 304 | 5.04 | 2.37 | 2.67 |
| 305 | 1.94 | 1.64 | 0.3 | 305 | 1.79 | 1.89 | −0.1 |
| 306 | 9.1 | 9.91 | −0.82 | 306 | 10.5 | 10.1 | 0.47 |
| 307 | 7.25 | 11.1 | −3.86 | 307 | 6.57 | 8.87 | −2.31 |
| 308 | 2.57 | 5.25 | −2.68 | 308 | 4.89 | 3.78 | 1.12 |
| 309 | 6.14 | 3.49 | 2.65 | 309 | 3.54 | 2.83 | 0.72 |
| 310 | 3.62 | 3.52 | 0.1 | 310 | 5.11 | 2.75 | 2.36 |
| 311 | 2.94 | 2.84 | 0.11 | 311 | 1.91 | 2.73 | −0.83 |
| 312 | 5.22 | 4.21 | 1.01 | 312 | 4.06 | 3.73 | 0.33 |
| 313 | 0.7 | 1.3 | −0.6 | 313 | 1.93 | 1.41 | 0.52 |
| 314 | 2.7 | 1.56 | 1.14 | 314 | 2.37 | 2.51 | −0.14 |
| 316 | 5.56 | 4 | 1.56 | 316 | 4.55 | 3.66 | 0.89 |
| 317 | 4.13 | 3.55 | 0.58 | 317 | 2.85 | 3.54 | −0.69 |
| 318 | 2.24 | 1.94 | 0.29 | 318 | 2.75 | 1.97 | 0.79 |
| 320 | 2.78 | 1.72 | 1.07 | 320 | 2.57 | 1.39 | 1.18 |
| 321 | 7.35 | 5.52 | 1.83 | 321 | 3.35 | 3.56 | −0.21 |
| 322 | 2.44 | 1.17 | 1.27 | 322 | 2.04 | 0.93 | 1.1 |
| 323 | 4.11 | 3.35 | 0.76 | 323 | 3.32 | 3.47 | −0.15 |
| 324 | 6.43 | 5.92 | 0.51 | 324 | 3.9 | 2.8 | 1.11 |
| 325 | 8.87 | 3.86 | 5.01 | 325 | 3.53 | 4.61 | −1.08 |
| 326 | 9.63 | 6.7 | 2.93 | 326 | 6.42 | 5.17 | 1.25 |
| 328 | 3.63 | 1.77 | 1.85 | 328 | 2.51 | 1.99 | 0.51 |
| 330 | 5.36 | 3.84 | 1.52 | 330 | 3.67 | 3.28 | 0.4 |
| 331 | 4.31 | 1.63 | 2.69 | 331 | 5.82 | 3.77 | 2.04 |
| 333 | 5.36 | 5.39 | −0.03 | 333 | 5.91 | 4.18 | 1.72 |
| 334 | 13.31 | 3.55 | 9.76 | 334 | 1.7 | 2.37 | −0.68 |
| 335 | 3.62 | 2.38 | 1.24 | 335 | 4.16 | 2.37 | 1.79 |
| 336 | 3.41 | 2.38 | 1.03 | 336 | 4.07 | 2.87 | 1.2 |
| Mean | 4.8 | 3.7 | 1.1 | Mean | 4.0 | 3.3 | 0.7 |
| STABW | 2.7 | 2.3 | 2.3 | STABW | 1.9 | 1.9 | 1.2 |
| t-test | | | 0.010 | t-test | | | 0.002 |

TABLE 2
Fasting insulin (with p-values absolute)

| Com- | | | | | | | |
|---|---|---|---|---|---|---|---|
| parison | | | | | | | |
| product | | Af- | Differ- | Vibamin | Be- | Af- | Differ- |
| Subject | Before | ter | ence | Subject | fore | ter | ence |
| 301 | 10.8 | 11.9 | −1.1 | 301 | 17.0 | 13.3 | 3.7 |
| 302 | 14.6 | 12.8 | 1.8 | 302 | 15.9 | 5.0 | 10.9 |
| 303 | 11.8 | 10.0 | 1.8 | 303 | 18.5 | 10.7 | 7.8 |
| 304 | 12.4 | 7.8 | 4.6 | 304 | 18.4 | 9.4 | 9.0 |
| 305 | 6.5 | 6.1 | 0.4 | 305 | 6.6 | 7.7 | −1.1 |
| 306 | 29.5 | 28.5 | 1.0 | 306 | 32.7 | 28.8 | 3.9 |
| 307 | 24.7 | 39.5 | −14.8 | 307 | 24.2 | 32.4 | −8.2 |
| 308 | 9.6 | 15.2 | −5.6 | 308 | 17.1 | 17.9 | −0.8 |
| 309 | 23.7 | 15.8 | 7.9 | 309 | 14.8 | 12.8 | 2.0 |
| 310 | 11.2 | 11.2 | 0.0 | 310 | 15.7 | 9.5 | 6.2 |
| 311 | 10.9 | 11.0 | −0.1 | 311 | 6.9 | 10.5 | −3.6 |
| 312 | 14.6 | 12.5 | 2.1 | 312 | 13.0 | 12.0 | 1.0 |
| 313 | 3.4 | 6.1 | −2.7 | 313 | 7.9 | 5.6 | 2.3 |
| 314 | 12.8 | 7.4 | 5.4 | 314 | 10.8 | 12.1 | −1.3 |
| 316 | 16.2 | 12.0 | 4.2 | 316 | 14.2 | 11.0 | 3.2 |
| 317 | 12.5 | 11.6 | 0.9 | 317 | 9.5 | 12.2 | −2.7 |
| 318 | 9.3 | 7.8 | 1.5 | 318 | 9.5 | 7.6 | 1.9 |
| 320 | 12.2 | 8.1 | 4.1 | 320 | 11.4 | 6.7 | 4.7 |
| 321 | 20.4 | 18.8 | 1.6 | 321 | 12.3 | 13.0 | −0.7 |
| 322 | 9.0 | 4.6 | 4.4 | 322 | 7.5 | 4.1 | 3.4 |
| 323 | 11.1 | 9.4 | 1.7 | 323 | 8.6 | 10.0 | −1.4 |
| 324 | 21.1 | 19.9 | 1.2 | 324 | 13.3 | 10.5 | 2.8 |
| 325 | 30.1 | 17.1 | 13.0 | 325 | 13.5 | 20.0 | −6.5 |
| 326 | 23.3 | 19.6 | 3.7 | 326 | 19.7 | 16.5 | 3.2 |
| 328 | 10.5 | 6.3 | 4.2 | 328 | 8.5 | 7.7 | 0.8 |
| 330 | 16.1 | 15.8 | 0.3 | 330 | 13.3 | 12.3 | 1.0 |
| 331 | 15.9 | 7.3 | 8.6 | 331 | 20.5 | 15.6 | 4.9 |
| 333 | 20.6 | 21.2 | −0.6 | 333 | 21.1 | 16.0 | 5.1 |
| 334 | 43.7 | 14.4 | 29.3 | 334 | 7.9 | 10.4 | −2.5 |
| 335 | 14.3 | 8.6 | 5.7 | 335 | 15.0 | 10.4 | 4.6 |
| 336 | 12.3 | 9.5 | 2.8 | 336 | 15.7 | 11.4 | 4.3 |
| Mean | 16.0 | 13.2 | 2.8 | Mean | 14.2 | 12.4 | 1.9 |
| STABW | 8.2 | 7.3 | 6.8 | STABW | 5.8 | 6.1 | 4.2 |
| t-test | | | 0.028 | t-test | | | 0.019 |

TABLE 3
Fasting glucose (with p-values absolute)

| Comparison | | | | | | | |
|---|---|---|---|---|---|---|---|
| product | | | | Vibamin | | | |
| Subject | Before | After | Difference | Subject | Before | After | Difference |
| 301 | 124 | 116 | 8.5 | 301 | 115 | 108 | 7.5 |
| 302 | 107 | 97 | 10 | 302 | 100 | 92.5 | 7.5 |
| 303 | 110.5 | 99 | 11.5 | 303 | 127 | 108 | 19 |
| 304 | 112.5 | 93 | 19.5 | 304 | 111 | 102 | 9 |
| 305 | 121 | 109 | 12 | 305 | 110 | 99.5 | 10.5 |
| 306 | 125 | 141 | −16 | 306 | 131 | 142 | −11 |
| 307 | 119 | 114 | 5 | 307 | 110 | 111 | −1 |
| 308 | 108.5 | 140 | −31.5 | 308 | 116 | 85.5 | 30.5 |
| 309 | 105 | 89.5 | 15.5 | 309 | 97 | 89.5 | 7.5 |
| 310 | 131 | 128 | 3.5 | 310 | 132 | 118 | 14.5 |
| 311 | 109.5 | 105 | 5 | 311 | 112 | 106 | 6.5 |
| 312 | 145 | 137 | 8.5 | 312 | 127 | 126 | 0.5 |
| 313 | 84 | 86.5 | −2.5 | 313 | 99 | 102 | −3 |
| 314 | 85.5 | 85.5 | 0 | 314 | 89 | 84 | 5 |
| 316 | 139 | 135 | 4 | 316 | 130 | 135 | −5 |
| 317 | 134 | 124 | 10 | 317 | 122 | 118 | 4 |
| 318 | 97.5 | 101 | −3.5 | 318 | 118 | 105 | 12.5 |
| 320 | 92.5 | 86 | 6.5 | 320 | 91.5 | 84 | 7.5 |
| 321 | 146 | 119 | 27 | 321 | 111 | 111 | −0.5 |
| 322 | 110 | 103 | 7 | 322 | 110 | 92 | 18 |
| 323 | 150 | 145 | 5.5 | 323 | 157 | 141 | 16 |
| 324 | 123.5 | 121 | 3 | 324 | 119 | 108 | 11 |
| 325 | 119.5 | 91.5 | 28 | 325 | 106 | 93.5 | 12.5 |
| 326 | 167.5 | 139 | 29 | 326 | 132 | 127 | 5 |
| 328 | 140 | 114 | 26 | 328 | 120 | 105 | 14.5 |
| 330 | 135 | 98.5 | 36.5 | 330 | 112 | 108 | 4 |
| 331 | 110 | 90.5 | 19.5 | 331 | 115 | 98 | 17 |
| 333 | 105.5 | 103 | 2.5 | 333 | 114 | 106 | 7.5 |
| 334 | 123.5 | 100 | 23.5 | 334 | 87 | 92.5 | −5.5 |
| 335 | 102.5 | 112 | −9.5 | 335 | 113 | 92.5 | 20 |
| 336 | 112.5 | 102 | 11 | 336 | 105 | 102 | 3 |
| Mean | 119 | 110 | 9 | Mean | 114 | 106 | 8 |
| STABW | 19 | 18 | 14 | STABW | 14 | 15 | 9 |
| t-test | | | 0.001 | t-test | | | 0.000021 |

TABLE 4
Weight (with p-values absolute)

| Comparison | | | | | | | |
|---|---|---|---|---|---|---|---|
| product | | | | Vibamin | | | |
| Subject | Before | After | Difference | Subject | Before | After | Difference |
| 301 | 106.6 | 103 | 3.4 | 301 | 106 | 104 | 1.2 |
| 302 | 92 | 87.1 | 4.9 | 302 | 85.4 | 82.7 | 2.7 |
| 303 | 98.7 | 96.5 | 2.2 | 303 | 99.9 | 97.9 | 2 |
| 304 | 93.9 | 90.6 | 3.3 | 304 | 93.4 | 92.4 | 1 |
| 305 | 94 | 89.5 | 4.5 | 305 | 90.5 | 90.5 | 0 |
| 306 | 130.4 | 127 | 3.7 | 306 | 128 | 128 | 0.1 |
| 307 | 106.6 | 103 | 3.2 | 307 | 109 | 106 | 2.8 |
| 308 | 116.4 | 113 | 3.3 | 308 | 115 | 114 | 1.3 |
| 309 | 107.7 | 104 | 3.3 | 309 | 106 | 108 | −2.6 |
| 310 | 97.6 | 95.3 | 2.3 | 310 | 95.7 | 93.7 | 2 |
| 311 | 90.6 | 91 | −0.4 | 311 | 92.6 | 89.7 | 2.9 |
| 312 | 80.2 | 79.4 | 0.8 | 312 | 79.8 | 78 | 1.8 |
| 313 | 108 | 106 | 2.4 | 313 | 114 | 107 | 7.5 |
| 314 | 107.7 | 105 | 2.9 | 314 | 111 | 106 | 4.9 |
| 316 | 84.5 | 80.5 | 4 | 316 | 80 | 77 | 3 |
| 317 | 96.8 | 93.7 | 3.1 | 317 | 92.8 | 90.8 | 2 |
| 318 | 85.3 | 80.8 | 4.5 | 318 | 91.3 | 85.7 | 5.6 |
| 320 | 88.4 | 82.1 | 6.3 | 320 | 81.8 | 78.9 | 2.9 |
| 321 | 95.8 | 91.4 | 4.4 | 321 | 91.1 | 87.8 | 3.3 |
| 322 | 96.5 | 91.1 | 5.4 | 322 | 92.9 | 88.8 | 4.1 |
| 323 | 108.7 | 105 | 3.5 | 323 | 106 | 102 | 4 |
| 324 | 109 | 104 | 4.8 | 324 | 106 | 105 | 1.3 |
| 325 | 110.8 | 106 | 5.2 | 325 | 108 | 104 | 4.4 |
| 326 | 125 | 123 | 1.7 | 326 | 123 | 122 | 0.8 |
| 328 | 78.2 | 75.3 | 2.9 | 328 | 75.5 | 72.6 | 2.9 |
| 330 | 99.9 | 96 | 3.9 | 330 | 95.6 | 92.4 | 3.2 |
| 331 | 93.6 | 91.9 | 1.7 | 331 | 95.7 | 92.6 | 3.1 |
| 333 | 98.9 | 98.2 | 0.7 | 333 | 98.2 | 99.9 | −1.7 |
| 334 | 85.5 | 80.5 | 5 | 334 | 79.5 | 77 | 2.5 |
| 335 | 69.8 | 68.4 | 1.4 | 335 | 72.7 | 68.4 | 4.3 |
| 336 | 105.1 | 105 | 0.6 | 336 | 109 | 105 | 4.2 |
| Mean | 98.8 | 95.6 | 3.2 | Mean | 97.5 | 95.0 | 2.5 |
| STABW | 13.2 | 13.2 | 1.6 | STABW | 13.7 | 14.1 | 2.0 |
| t-test | | | 0.00000000 | t-test | | | 0.0000001333 |

TABLE 5
Significance values after elimination of interfering factors (“weight”)

| | | With adjustment |
|---|---|---|
| “Vibamin ®” | Without adjustment | (“weight”) |
| Weight | <0.001 | |
| Blood sugar | <0.001 | 0.005 |
| Insulin | 0.019 | 0.030 |
| HOMA-IR | 0.002 | 0.007 |
| Comparison product | | Weight |
| Weight | <0.001 | |
| Blood sugar | 0.001 | 0.3309 |
| Insulin | 0.028 | 0.9042 |
| HOMA-IR | 0.010 | 0.9167 |

1. The use of distillers grains which, in addition to a fermentation with yeast, were also fermented with a yogurt culture and/or buttermaking culture, for producing a composition for preventing and/or treating increased blood sugar values.

2. The use as claimed in claim 1, wherein the increased blood sugar values are selected from fasting and/or postprandial blood sugar values.

3. The use as claimed in claim 1, wherein use is made of distillers grains having a residual starch content of a maximum of 4% by weight, based on the total weight of the distillers grains.

4. The use as claimed in claim 1 for producing a composition for preventing, and/or treating patients having, prediabetes and/or diabetes and/or a predisposition thereto, and associated forms and/or preforms.

5. The use as claimed in claim 4, wherein diabetes is selected from diabetes type Ia and Ib, type IIa and IIb and/or autosomally inherited diabetes of youth, and wherein the associated forms and/or preforms are selected from secondary diabetes forms, potential diabetes, latent diabetes, decreased glucose tolerance and/or clinically manifests diabetes.

6. The use as claimed in claim 5, wherein diabetes is type II diabetes.

7. The use as claimed in claim 1 for producing a composition for reducing body weight.

8. The use as claimed in claim 1 for producing a composition for preventing, and/or treating patients having, adiposity.

9. The use as claimed in claim 8 for producing a composition for preventing, and/or treating patients having, adiposity, and simultaneously increased fasting blood sugar values.

10. The use as claimed in claim 1 for producing a composition for preventing, and/or treating patients having, metabolic syndrome.

11. The use as claimed in claim 10 for producing a composition for preventing, and/or treating patients having, insulin resistance, arteriosclerosis, lipid, protein and/or carbohydrate metabolic disorders.

12. The use as claimed in claim 1, wherein the distillers grains have a low glycemic index.

13. The use as claimed in claim 1, wherein distillers grains are used which were produced by the distillers grains being taken off directly from the still, then thickened, but not dried, and subsequently being fermented with milk and a yogurt culture and/or buttermaking culture.

14. The use as claimed in claim 13, where distillers grains are used which were produced by.
a) the distillers grains being thickened to approximately 15 to 17% dry mass at a pressure of 100 to 800 mbar, then
b) being mixed with a milk already inoculated with the yogurt culture and/or buttermaking culture,
c) this mixture either being fermented with a yogurt culture for 10 to 20 hours at a temperature of 38 to 48° C., or
this mixture being fermented with a buttermaking culture for up to 36 hours at a temperature of 18 to 24° C., and thereafter
d) cooled, mixed with further milk or milk concentrate, to temperatures below 15° C., and
e) which is optionally subsequently spray dried.

15. The use as claimed in claim 1, wherein use is made of distillers grains based on wheat, barley, rye, oats, corn and/or rice.

16. The use as claimed in claim 1, wherein the distillers grains are administered in a daily dose of 100 to 400 g.

17. The use as claimed in claim 16, wherein the distillers grains are administered in 2 to 5 individual doses per day.

18. The use as claimed in claim 17, wherein the distillers grains are administered in equally sized individual doses.

19. The use as claimed in claim 1, wherein the distillers grains are administered in viscous form.

20. The use as claimed in claim 19, wherein the distillers grains are administered in water or fermented milk.

21. The use as claimed in claim 19, wherein the distillers grains are administered in curdled milk, buttermilk, yogurt, quark or kefir.

22. The use as claimed in claim 1, wherein the distiller grains are administered as a meal replacement and/or meal supplement.

23. The use as claimed in claim 22, wherein the distillers grains are administered over a period of at least 3 successive weeks.

24. The use as claimed in claim 1, wherein use is made of the distillers grains in combination and/or together with further anti-diabetes acting or diabetes-preventing active ingredients.

25. The use as claimed in claim 24, wherein use is made of the distillers grains in combination and/or together with further active ingredients counteracting or preventing type II diabetes.

26. The use as claimed in claim 24, wherein use is made of the distillers grains in the form of, and/or in combination with, and/or together with, food supplements, functional food and/or special diet.

27. The use as claimed in claim 24, wherein use is made of the distillers grains in the form of, and/or in combination with, and/or together with, creatine, creatine monohydrate, a-lipoic acid, phytosterol, polyunsaturated fatty acids and/or phospholipids and also their derivatives and/or mixtures.

28. The use as claimed in claim 1, for producing a composition having a regulating effect on the intestinal flora.

29. The use as claimed in claim 28, wherein the dietary fibers of the distillers grains exhibit this activity.

30. The use as claimed in claim 28, wherein the distillers grains are administered as a meal replacement and/or meal supplement.

31. The use as claimed in claim 28, wherein the distillers grains are administered with sufficient liquid.
